(19) United States
(12) Patent Application Publication
(10) Pub. No.: US 2010/0255099 A1
(43) Pub. Date: Oct. 7, 2010

(54) CLAVULANATE FORMULATION FOR NEUROPROTECTION AND TREATMENT OF NEURODEGENERATIVE DISORDERS

(75) Inventors: Deog J. Kim, Rockville, MD (US); Young B. Lee, Clarksburg, MD (US); Chang H. Ahn, Potomac, MD (US); Edward C. Scholtz, Blue Bell, PA (US); Youngbuhm Huh, Seoul (KR)

Correspondence Address: VENABLE LLP, P.O. BOX 34385, WASHINGTON, DC, 20043-9998, US

(73) Assignee: REXAHN PHARMACEUTICALS, INC., Rockville, MD (US)

(21) Appl. No.: 12/770,304

(22) Filed: Apr. 29, 2010

Related U.S. Application Data
(63) Continuation-in-part of application No. 12/258,062, filed on Oct. 24, 2008.
(60) Provisional application No. 60/996,079, filed on Oct. 26, 2007.
(60) Provisional application No. 61/173,841, filed on Apr. 29, 2009.

(51) Int. Cl. A61K 31/424; A61P 25/28; A61P 25/16; A61K 9/14

(52) U.S. Cl. 424/487; 514/210.06; 424/488

(57) ABSTRACT

The present invention generally relates to use of a stable solid pharmaceutical compositions that includes a clavulanate as the pharmaceutically active ingredients in an immediate-release or an extended-release solid dosage form. The composition can be used in a method of treating a neurodegenerative disease, providing neuroprotection, or preventing neuronal cell loss or death. Exemplary neurodegenerative diseases include Parkinson's disease, Alzheimer's disease and multiple sclerosis.

This application claims priority to U.S. Provisional Application No. 61/173,841, filed Apr. 29, 2009, the contents of which are hereby incorporated by reference in their entirety.

### FIELD OF THE INVENTION

The present invention relates to uses of stable solid oral dosage forms comprising clavulanic acid, pharmaceutically acceptable clavulanic acid salts, salt compositions and derivatives. In particular, the present invention provides the use of immediate release compositions and extended release compositions of potassium clavulanate that are suitable for daily use and which achieve therapeutic levels of clavulanate for neuroprotection and for treatment of neurodegenerative disorders.

### BACKGROUND OF THE INVENTION

The name of clavulanic acid is derived from the Streptomyces clavuligerus microorganisms from which clavulanic acid is derived. Clavulanic acid is biosynthetically generated from the amino acid arginine and the sugar glyceraldehyde 3-phosphate.

Clavulanic acid has negligible intrinsic antimicrobial activity, despite sharing the β-lactam ring that is characteristic of β-lactam antibiotics. However, the similarity in chemical structure allows the molecule to act as a competitive inhibitor of β-lactamases secreted by certain bacteria to confer resistance to β-lactam antibiotics. When given in combination with some β-lactam antibiotics like ticarcillin or amoxicillin, clavulanic acid can extend the spectrum and enhance the activity of the antibiotic (AHFS, 1991). This synergistic activity is possible because clavulanic acid acts as an irreversible competitive inhibitor of bacterial β-lactamases that naturally degrade and inactivate β-lactam antibiotics (Brown et al., J Antibiot (Tokyo). 1976, 29:668-669; Reading and Cole, Antimicrob Agents Chemother. 1977, 11:852-857).

In addition to its inhibitory effect on β-lactamases, clavulanic acid has shown effectiveness for neuroprotection, and in treating anxiety and sexual dysfunction. Several mechanisms have been proposed for the neuroprotective and neurological activity of clavulanic acid. Koppel et al., in U.S. Pat. Nos. 6,489,319; 6,610,681; and 6,627,625, each of which is incorporated herein by reference in its entirety, describe that clavulanic acid itself has an anxiolytic activity when administered i.p. at less than 1 μg/kg. U.S. Pat. No. 6,426,342, which is incorporated herein by reference in its entirety, describes the potent neuroprotectant activity of clavulanic acid when treated rats with clavulanic acid at an i.p. dose of 1 μg/kg. U.S. Pat. No. 7,166,626, which is incorporated herein by reference in its entirety, discloses a method for treating sexual dysfunction with the administration of clavulanic acid. U.S. Pat. No. 6,489,319 reports that clavulanic acid could alter CNS activity and behavior at doses ranging from 10 ng to 10 μg/kg. Thus the unique neurological activity profiles of clavulanic acid provide strong evidence that the compound interacts with unique sets of neurogenic targets. Rothstein et al also demonstrated that several β-lactam antibiotics could offer neuroprotection by the activation of the gene for glutamate neurotransmitter transporter (Nature, 2005, 433:73-77). Since first identified with the discovery of penicillin in 1928, β-lactam antibiotics have been among the most widely used antibiotics, and have not shown substantial toxic CNS actions at normal antibacterial doses. Therefore, β-lactam antibiotics may be used as a new and safe therapeutic agent for the treatment of CNS related diseases.

The instability of many of dry formulations containing clavulanic acid and derivatives or salts thereof (collectively referred to as clavulanate) has necessitated the inclusion of a complex formulation of excipients, including binders, glidants, disintegrants and even desiccants, etc. to yield a pharmaceutically acceptable carrier. This is in part due to the fact that clavulanate is a highly hygroscopic material which is highly unstable in aqueous media. Methods of formulation must therefore ensure that the product can retain its potency during storage, and yet can subsequently yield satisfactory dissolution rates. One such process is disclosed in WO 92/19227, incorporated herein by reference in its entirety, and mandates the inclusion of both an intra-cellular and an extra-cellular disintegrant. Another process described in U.S. Pat. No. 4,537,887, incorporated herein by reference in its entirety, specifies the inclusion of an edible desiccant within the composition itself. Other processes warrant the inclusion of a desiccant within a container housing the amoxicillin/clavulanate combination. In this regard, U.S. Pat. Nos. 4,301,149 and 4,441,609 which are incorporated herein by reference in their entirety are particularly salient. Potassium clavulanate is more stable than the free acid and the least hygroscopic of the pharmaceutically acceptable clavulanic acid salts, and it is therefore most frequently used for commercial preparations. However, potassium clavulanate is still extremely hygroscopic and susceptible to hydrolysis so that co-amoxicillin/clavulanate formulations are prone to degradation on storage even under low humidity conditions. The presence of water in crystallization of amoxicillin may contribute to instability of these dosage forms, accelerating the decomposition of clavulanate once any degradation has commenced.

### SUMMARY OF THE INVENTION

Clavulanate is an exceptionally difficult material to formulate because of its moisture and heat sensitivity. There is a need to develop stable solid formulations of clavulanate alone, i.e. without anti-biotics, especially at low doses such as 10 μg to 10 mg, for example, from about 0.1 mg to about 5 mg, which is orally active in order to provide neuroprotection or for the treatment of neurodegenerative disorders.

The present invention is a method for providing neuroprotection and for treating a neurodegenerative disease comprising orally administering a stable oral dosage composition containing clavulanate, in the form of an immediate release composition or an extended release composition. The dosage form can be prepared from clavulanic acid or derivatives or salts thereof, for example potassium clavulanate or Clavitesse™, that is suitable for daily use.

The present invention overcomes and alleviates the above mentioned drawbacks and disadvantages through the development of stable oral clavulanate pharmaceutical compositions and methods for providing neuroprotection and for treating a neurodegenerative disease using the composition. Generally speaking, the present invention relates to uses of stable solid pharmaceutical compositions, and in particular immediate release or extended release compositions, that include clavulanate as the pharmaceutically active ingredient. The pharmaceutical compositions can be provided in a solid dosage form, such as a tablet, capsule, pill, troche or powder. The solid pharmaceutical composition can include a clavulanate in the presence of one or more pharmaceutically acceptable excipients, where the clavulanate is present in an amount of between about 10 μg and about 10 mg or, for example, from about 0.1 mg to about 5 mg. The composition can provide a therapeutically useful amount of clavulanate upon administration. Examples of clavulanates include clavulanic acid, clavulanic acid derivatives and pharmaceutically acceptable salts of clavulanic acid. The clavulanate can be present in an amount between about 0.01% and about 10% by weight of the composition. In some embodiments, the moisture content of the composition is less than about 4% of the total weight. The formulation is the form of a tablet, capsule, pill, troche or powder. Exemplary solid pharmaceutical compositions according to the invention can have a moisture content of less than 10% after storage at 25° C. and 60% relative humidity or after storage at 30° C. at 65% relative humidity for three months.

In exemplary compositions, the clavulanate is potassium clavulanate. The potassium clavulanate can be provided as, for example, a powder or as a 1:1 mixture with silicon dioxide or microcrystalline cellulose. Exemplary compositions are immediate-release compositions which release more than 80% of clavulanate from the tablet within approximately 5 to approximately 30 minutes after administration. In exemplary embodiments, the composition is prepared by a method where potassium clavulanate powder is lyophilized in the presence of the one or more pharmaceutically acceptable excipients. In an example of an immediate release composition, the composition can contain from about 10% to about 20% by weight of a binder or diluent, about 45% to about 55% by weight of a filler, about 20% to about 40% by weight of a disintegrant and about 3% to about 6% by weight of a lubricant. In a such an embodiment, an exemplary binder or diluent is Maltrin M150, an exemplary filler is Prosolve SMCC 50, an exemplary disintegrant is Pharmaburst and/or L HPC LH-11 and/or Acdisol and an exemplary lubricant is stearic acid.

In other exemplary embodiments, the composition is prepared by a method where potassium clavulanate in a 1:1 mixture with silicon dioxide or microcrystalline cellulose is lyophilized in the presence of the one or more pharmaceutically acceptable excipients. In another example of an immediate release composition, the composition can contain from about 50-60% of a filler, about 20-30% of a disintegrant, about 0.5-5% of a flow enhancer/moisture protectant and/or about 3-6% of a lubricant. In a such an embodiment, an exemplary filler is Prosolve SMCC 50, an exemplary disintegrant is Pharmaburst and/or Acdisol, an exemplary flow enhancer/moisture protectant is Carbosil and an exemplary lubricant is magnesium stearate.

In another embodiment, the pharmaceutical composition is an extended-release composition which releases the potassium clavulanate over at least about 4 hours. An extended release composition can be prepared where a potassium clavulanate powder or a potassium clavulanate in a 1:1 mixture with microcrystalline cellulose is lyophilized in the presence of the one or more pharmaceutically acceptable excipients. Exemplary excipients can include one or more of a matrix, a filler, a glidant and a lubricant. In an example of an extended release composition, the composition can contain from about 20% to about 40% by weight of a matrix, about 50% to about 75% by weight of a filler, about 0.1% to about 1% by weight of a glidant and about 1% to about 2% by weight of a lubricant. In such an embodiment, exemplary matrices are Klucel LF, Methocel K100LV Prem CR, Eudragit S100, Carbopol 971P, Carbopol 974P, methacrylate copolymer type A and methacrylate copolymer type B and mixtures thereof; exemplary fillers are anhydrous lactose, Avicel PH-112, Avicel PH-113, Isomalt, or mixtures thereof; an exemplary glidant is Carbosil and an exemplary lubricant is at least one of magnesium stearate and talc.

In other embodiments, a solid pharmaceutical dosage form for use in methods of the present invention is prepared by providing a clavulanate such as clavulanic acid, clavulanic acid derivatives or a pharmaceutically acceptable salt of clavulanic acid; mixing the clavulanate with at least one excipient; granulating the mixture of clavulanate and the at least one excipient; and lyophilizing the granulated mixture of clavulanate and the at least one excipient. The granulating step can be, for example wet granulation. An exemplary clavulanate is potassium clavulanate, for example in the form of potassium clavulanate powder or potassium clavulanate as a 1:1 mixture with silicon dioxide or microcrystalline cellulose. In an exemplary method, the excipient at least one of a binder, a diluent, a filler, a disintegrant, a matrix, a filler, a glidant, a flow enhancer, a moisture protectant, and a lubricant. The method can include forming the dosage form into a tablet or bead, and optionally coating the tablet or beads with a delay-release polymer. The invention includes orally administering a stable solid pharmaceutical composition according to the invention to provide an amount of clavulanate effective for neuroprotection or for the treatment of a neurodegenerative disorder such as Parkinson's disease, Alzheimer's disease or multiple sclerosis.

Still other embodiments of the present invention relate to the use of immediate and extended release formulations of clavulanate that are suitable for oral administration.

Yet other embodiments of the present invention relate to a freeze drying method for preparing the pharmaceutical formulation, wherein the freeze drying comprises the drying process to dehydrate the hydrated pharmaceutical composition.

Other embodiments of the invention relate to a processes for the preparation of pharmaceutical compositions containing clavulanate and to their use as medicaments.

In other embodiments, the invention is a method of treating a neurodegenerative disease by orally administering a stable oral formulation that includes a therapeutically effective amount of a clavulanate, such as clavulanic acid, a clavulanic acid derivative or a pharmaceutically acceptable salt of clavulanic acid. Another exemplary embodiment is a method of providing neuroprotection comprising orally administering a stable oral formulation containing a clavulanate. Neuroprotection includes preventing cell loss or cell death from a neurodegenerative disease. Yet another embodiment is a method of preventing neuronal cell loss or death comprising orally administering a stable oral formulation of a clavulanate. Examples of neurodegenerative diseases treatable according to methods of the invention include Parkinson's disease, Alzheimer's disease, and multiple sclerosis. Treating can include, for example, reducing the frequency, onset time or severity of seizures or tremors; reducing memory loss; or reducing neuronal cell death.

In exemplary methods according to the invention, the clavulanate is potassium clavulanate. The stable oral formulation can be in the form of a tablet, capsule, pill, troche, solution, suspension, buccal or sublingual tablet, orally disintegrating tablet, thin film or powder. The formulation can be an extended-release composition which releases the clavulanate for at least about 4 hours; an immediate-release composition which releases the clavulanate in less than about 0.5 hours; or other forms. In some embodiments, the potassium clavulanate is potassium clavulanate powder or potassium clavulanate as a 1:1 mixture with silicon dioxide or microcrystalline cellulose. Formulations useful with the invention can include one or more of a matrix; a filler; a glidant; and a lubricant. The matrix can be, for example, Methocel K100LV Prem CR, Eudragit S100, Carbopol 971P, Carbopol 974P, methacrylate copolymer type A, methacrylate copolymer type B or mixtures thereof. The filler can be, for example, anhydrous lactose, Avicel PH-112, Avicel PH-113, Isomalt, or mixtures thereof. The glidant can be, for example, Carbosil and exemplary lubricants are magnesium stearate, talc and mixtures thereof.

An exemplary method of preparing a formulation for use in a method of the invention includes mixing the clavulanate with at least one excipient; granulating the mixture of clavulanate and the at least one excipient; and lyophilizing the granulated mixture of clavulanate and the at least one excipient.

According to the invention, the formulation can be administered in an amount that provides from about 0.001 mg/kg/day to about 1.0 mg/kg/day of clavulanate. In some embodiments, the formulation can be administered in an amount that provides from about 0.01 mg/kg/day to about 1.0 mg/kg/day. The formulation may be administered in a single daily dose or in multiple doses.

### BRIEF DESCRIPTION OF THE DRAWINGS

FIG. 1 shows in vitro dissolution profiles of clavulanate immediate release formulation, Sample B () and C(◯).

FIG. 2 shows in vitro dissolution profiles of clavulanate extended release formulation, Sample F.

FIG. 3 shows in vitro dissolution profiles of clavulanate extended-release formulation, Sample I.

FIG. 4 illustrates the stability of Sample D (5 mg/tablet of 1:1 mixture of potassium clavulanate and microcrystalline cellulose) at 25° C./60% humidity () and 30° C./65% humidity (▴).

FIG. 5 illustrates the stability of Sample E (5 mg/tablet of 1:1 mixture of potassium clavulanate and silicon dioxide) at 25° C./60% humidity () and 30° C./65% humidity (▴).

FIG. 6 illustrates the stability of Sample F (5 mg/tablet of 1:1 mixture of potassium clavulanate and microcrystalline cellulose) at 2-8° C. (0), 25° C./60% humidity () and 30° C./65% humidity (▴).

FIG. 7 illustrates the stability of Sample G (5 mg/tablet) at 2-8° C. (0), 25° C./60% humidity () and 30° C./65% humidity (▴).

FIG. 8 shows the immunohistochemistry for tyrosine hydroxylase (TH) in the substantia nigra pars compacta (SNpc). The number of TH-positive neurons were significantly decreased in MPTP (1-methyl-4-phenyl-1,2,3,6-tetrahydropyridine)-saline group compared to normal group. The number of TH-positive neurons were well preserved in MPTP-Clavulanate treatment group.

FIG. 9 shows the effects of Clavulanate treatment on substantia nigra pars compacta (SNpc) neuron survival in MPTP (1-methyl-4-phenyl-1,2,3,6-tetrahydropyridine)-treated animals.

FIG. 10 illustrates the behavioral effects of Clavulanate on MPTP (1-methyl-4-phenyl-1,2,3,6-tetrahydropyridine)-induced neurotoxicity using pole test in mouse PD model.

FIG. 11 shows the effect of Clavulanate on kainate (KA) induced hippocampal neurotoxicity in the CA3 region.

FIG. 12 shows the results of cresyl violet staining in the CA3 region in normal, kainate+saline, and kainate+Clavulanate treated groups.

### DETAILED DESCRIPTION OF THE INVENTION

As used herein, the term clavulanate herein includes clavulanic acid (I), pharmaceutically acceptable clavulanic acid salts, salt compositions and derivatives, such as esters. An example of pharmaceutically acceptable clavulanic acid salts is potassium clavulanate. Potassium clavulanate may be supplied as a pure compound or as, for example, Clavitesse™, a 1:1 mixture of potassium clavulanate and microcrystalline cellulose or a 1:1 mixture of potassium clavulanate and silicon dioxide (available from DSM Anti-Infectives B.V., The Netherlands).

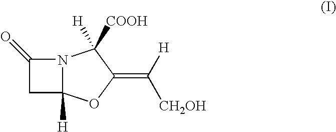

Exemplary derivatives include active esters of clavulanic acid, for example, acyloxyalkyl groups such as acetoxymethyl, pivaloyloxymethyl, β-acetoxyethyl, β-pivaloyloxyethyl, 1-(cyclohexylcarboonyloxy) prop-1-yl, and (1-aminoethyl) carbonyloxymethyl; alkoxycarbonyloxyalkyl groups, such as ethoxycarbonyloxymethyl and alpha-ethoxycarbonyloxyethyl; dialkylaminoalkyl groups, such as ethoxycarbonyloxymethyl and β-ethoxycarbonyloxyethyl; dialkylaminoalkyl especially di-lower alkylamino alkyl groups such as dimethylaminomethyl, dimethylaminoethyl, diethylaminomethyl or diethylaminoethyl-2-(alkoxycarbonyl)-2-alkenyl groups such as 2-(isobutoxycarbonyl) pent-2-enyl and 2-(ethoxycarbonyl)but-2-enyl; lactone groups such as phthalidyl and dimethoxyphthalidyl.

Exemplary salts include any pharmaceutically acceptable salt of clavulanic acid, for example, aluminum, alkali metal salts such as sodium or potassium, alkaline earth metal salts such as calcium or magnesium, and ammonium or substituted ammonium salts, for example those with lower alkylamines such as triethylamine, hydroxy-lower alkylamines such as 2-hydroxyethylidene, bis-(2-hydroxyethyl)amine or tris-(2-hydroxyethyl)amine, cycloalkylamines such as dicyclohexylamine, or with procaine, dibenzylamine, N,N-dibenzylethylenediamine, 1-ephenamine, N-methylmorpholine, N-ethylpiperidine, N-benzyl-β-phenethylamine, dehydroabietylamine, N,N′-bisdehydro-abietylamine, ethylenediamine, or bases of the pyridine type such as pyridine, collidine or quinoline, or other amines, lithium salt and silver salt.

The term “oral administration” as used herein includes any form of delivery of a therapeutic agent or a composition thereof to a subject wherein the agent or composition is placed in the mouth of the subject, whether or not the agent or composition is swallowed. Thus “oral administration” includes buccal and sublingual as well as esophageal administration. Absorption of the agent can occur in any part or parts of the gastrointestinal tract including the mouth, esophagus, stomach, duodenum, ileum and colon.

As used herein, a “subject” to which a therapeutic agent or composition thereof can be administered includes a human patient of either sex and of any age, and also includes any nonhuman animal, particularly a domestic or companion animal, illustratively a cat, dog or horse.

The term “neurodegenerative disorder” refers to conditions, disorders, and/or diseases that are associated with degeneration, whole or partial loss of function, or irregular function of the nervous system. Thus, any condition, disorder and/or disease that affects any component or aspect of the nervous system (either central or peripheral) in such a way can be considered a neurodegenerative disorder. neurodegenerative disorder includes, but is not limited to cognitive disorders, movement disorders, mental disorders, pain disorders, sleep disorders, etc. Exemplary neurodegenerative disorders include, but are not limited to Parkinson's disease, Alzheimer's disease and multiple sclerosis. Exemplary movement disorders can include various dyskinesias such as tremor, dystonia, chorea, athetosis, tic disorders, blepharospasm, as well as hemiballysmus, myoclonus, focal dystonias, such as writer's cramp and torticollis, restless leg syndrome and asterixis. These excessive or otherwise abnormal involuntary movements may vary significantly in rate, frequency, periodicity and progressionary character. Such movements may be seen in sometimes overlapping disorders such as Parkinson's disease; essential tremor, a.k.a. benign tremor or familial tremor; tic disorders, e.g. Tourette's syndrome; idiopathic dystonia (inducing writer's cramp), progressive supranuclear palsy and Wilson's disease.

As used herein, the terms “treat,” “treatment,” etc. refer any detectable, clinically significant improvement, delay in the onset, prevention of the onset, or amelioration of the disorder or any symptoms of a disorder or condition. Treatment does not require or demand a cure.

“Neuroprotection” refers in particular to methods that delay or prevent the onset of a neurodegenerative or neurological disorder, i.e. a disorder affecting the neurological or nervous system, including the central or peripheral nervous system. Neuroprotection or neuroprotective effects can be measured empirically, for example, by behavioral or cognitive changes or lack thereof, physiologically, for example by showing a preservation or lack of or reduction of destruction of neurons or neuronal death as compared to untreated controls, or any other metric that measures the lack of an adverse effect on any part of the neurological system. Exemplary locations where neuronal survival or lack of reduction can be demonstrated include the substantia nigra pars compacta (SNpc) and the hippocampal CA3 region.

The term “excipient” as used herein means any substance, not itself a therapeutic agent, used as a carrier or vehicle for delivery of a therapeutic agent to a subject or added to a pharmaceutical composition to improve its processing, handling, storage, disintegration, dispersion, dissolution, release or organoleptic properties or to permit or facilitate formation of a dose unit of the composition into a discrete article such as a capsule or tablet suitable for oral administration. Excipients can include, by way of illustration and not limitation, diluents, disintegrants, binding agents, adhesives, wetting agents, polymers, lubricants, glidants, substances added to mask or counteract a disagreeable taste or odor, flavors, dyes, fragrances, and substances added to improve appearance of the composition.

The present invention is thus directed to use of an immediate or extended release formulation of potassium clavulanate or Clavitesse™ which is suitable for oral administration. The formulations of the present invention comprise a quantity of a quick release preparation of clavulanate or a quantity of a slow release (or extended release) preparation of clavulanate. An immediate release formulation is characterized by its rapid release of clavulanate, the rapid release characterized by obtaining a maximal release of clavulanate within approximately 5 to approximately 30 minutes after administration. An extended release formulation is characterized by a slower release of clavulanate over, for example, at least about 4 hours. In exemplary embodiments, the extended release formulation can release clavulanate over at least about 6 or at least about 8 hours. These or other embodiments can continue to release clavulanate after initial administration for at least about 3 hours, at least about 4 hours, at least about 5 hours, at least about 6 hours, at least about 7 hours, or at least about 8 hours. In an exemplary embodiment, the present invention is a tablet or a capsule containing the immediate or extended release formulation, which, based upon the total quantity of drug in the formulation rather than total weight of the formulation, comprises the amount of active compound from about 10 μg to 10 mg or about 0.01% to 10% of total weight of the active compound.

Neuroprotection and treatment of neurodegenerative disorders according to the present invention can be achieved by orally administering a stable, solid formulation of a clavulanate. Treatment can be evaluated in a number of ways, including subject survival, behavioral testing, immunohistological evaluation, for example measuring cellular or neuronal survival, or measuring the frequency or intensity of a particular symptom indicative of a disorder. In animal models or human studies, behavioral testing can include, for example, evaluation of the ability to orient oneself, motor impairment as evaluated with respect to, for example, speed and direction, and the like. Behavioral testing can also include testing memory through the use of mazes, for example the Morris water maze test, and the like. Immunohistological evaluation can be carried out on stained free floating sections followed by cell counting or other techniques generally known in the art. Symptomatic testing can include, for example, evaluating the number, frequency or intensity of various symptoms, for example seizures or tremors, or by evaluating retention of memory.

According to the present invention, it has been found that, in a MPTP (1-methyl-4-phenyl-1,2,3,6-tetrahydropyridine)-induced Parkinson's disease animal model, there is a preservation of the number of TH (tyrosine hydroxylase)-positive neurons in a group pre-treated by oral administration of stable solid clavulanate as compared to MPTP-saline group. Further, animals pre-treated with clavulanate showed a significant neuroprotective effect on kainate induced hippocampal cell death. Treated animals also showed a longer onset to seizure and mild seizure activity as compared to controls.

In some embodiments for neuroprotection or treatment of neurodegenerative diseases, a stable solid formulation can be administered in an amount that provides about 0.001 mg/kg to about 1.0 mg/kg clavulanate. In some embodiments, a stable solid formulation can be administered in an amount that provides about 0.01 mg/kg to about 1.0 mg/kg clavulanate. In some embodiments, a stable solid formulation can be administered in an amount that provides about 0.1 mg/kg to about 1.0 mg/kg clavulanate. Such dosages can be administered once per day, twice per day, three times per day, or more as indicated by evaluation of efficacy. Dosage forms can be can be formulated for administration in any suitable and convenient unit amount, for example, 0.1 mg per dose, 0.5 mg per dose, 1.0 mg per dose, 5 mg per dose, etc. In an exemplary embodiment, the stable solid formulation includes about 5 mg clavulanate per dose.

A daily dose can be administered in a single dosage or be divided into multiple doses to be administered over the course of a day. The multiple dose can be two, three, four or more doses per day. As will be appreciated, extended release compositions can provide a means for lowering the total number of daily doses that must be taken while delivering a similar total daily dose. A stable solid dosage form is particularly advantageous for use in the present invention as it can assure that under-dosing or over-dosing is less prevalent. This is particularly significant in the present application where even small amounts of decomposition in absolute terms can result in a relatively large change in percentage of clavulanate actually administered. In some prior uses of clavulanate, for example use in as an auxiliary agent in conjunction with antibiotics as a β-lactamase inhibitor, the lack of stability can be addressed by using excess clavulanate, so that decomposition has less effect on efficacy. However, in applications such as described herein where clavulanate is the active pharmaceutical ingredient, it is more important that a practitioner be able to administer clavulanate in a predictable predetermined quantity suitable for therapeutic purposes. This can further increase the efficacy of treatment as well as patient compliance.

The oral administration of pharmaceutical agents, such as tablets or capsules has certain advantages over parenteral administration such as i.v. or i.m. Diseases requiring treatment with painful injectable formulations are considered to be more serious than those conditions which can be treated with oral dosage forms. However, the major advantage with oral formulations is held to be their suitability for self administration whereas parenteral formulations have to be administered in most cases by a physician or paramedical personnel. For the present invention, oral administration is shown to have increased efficacy with respect to at least some indicators of neurodegenerative disorders.

The nature of various drug substances, e.g., particle size distribution, bulk density, flowability, wetting behavior, surface area and sticking tendency, varies greatly and can effect the processability of a solid dosage form such as a tablet. Clavulanate is highly hygroscopic and, upon contact with water, changes from a crystalline state to an amorphous state, which shows inferior stability. The combination of these hurdles makes standard tablet manufacturing processes extremely difficult, makes storage of clavulanate formulations problematic, and has resulted in special conditions for storage and preparation of formulations containing clavulanate.

Potassium clavulanate, although the most common and easily handled form, remains an exceptionally difficult material to formulate, being extremely hygroscopic and moisture sensitive. Degradation readily occurs in the presence of water and aqueous media. In applications such as described herein where clavulanate is the active pharmaceutical ingredient, it is more important that a practitioner be able to administer clavulanate in a predictable predetermined quantity suitable for therapeutic purposes. Administering a stable oral dosage form is desirable for therapeutic uses in these cases.

Accordingly, a suitable and robust clavulanate formulation overcoming the above problems that takes into account the properties of clavulanate is needed for neuroprotection and treatment of neurodegenerative disorders where clavulanate is the sole active ingredient. The problems encountered with clavulanate formulations are particularly challenging in the case of formulations at low dosages such as 10 μg to 10 mg where even a small degree of degradation can lead to a dramatic change in the amount of clavulanate available to a subject.

The present invention relates preparations of the stable oral dosage forms of clavulanate and use thereof for neuroprotection and in the treatment of neurodegenerative disorders. Solid oral dosage forms for use according to the invention can comprise additives or excipients that are generally suitable for the preparation of the solid oral dosage form. Solid oral dosage forms include, for example, tablets, capsules, pills, troches and powders. In the case of capsules, the solid oral dosage form can be a bead within a capsule. In exemplary embodiments of the invention, the solid oral dosage form is a stable solid tablet.

Tabletting aids, commonly used in tablet formulation can be used and reference is made to the extensive literature on the subject, see in particular Fiedler's “Lexicon der Hilfstoffe”, 4th Edition, ECV Aulendorf 1996, which is incorporated herein by reference. These include, but are not limited to, fillers, binders, disintegrants, lubricants, glidants, stabilizing agents, fillers or diluents, surfactants, film formers, softeners, pigments and the like.

Fillers include starches, e.g., potato starch, wheat starch, corn starch, hydroxypropyl cellulose, hydroxyethyl cellulose, hydroxypropyl methyl cellulose (HPMC) and, microcrystalline cellulose, e.g., products available under the registered trade marks AVICEL, FILTRAK, HEWETEN, Prosolve SMCC50 or PHARMACEL. Other examples of fillers include maltose, isomalt, lactose (for example as Pharmatose®), sucrose, glucose, mannitol, sorbitol, and calcium carbonate.

Binders include starches, sugars, cellulose or modified cellulose such as hydroxypropyl cellulose, lactose, or sugar alcohols like xylitol, sorbitol or maltitol. An exemplary binder is maltodextrin (Maltrin M150).

As disintegrants one can mention carboxymethylcellulose calcium (CMC-Ca), carboxymethylcellulose sodium (CMC-Na), crosslinked PVP (e.g. CROSPOVIDONE, POLYPLASDONE or KOLLIDON XL), alginic acid, sodium alginate and guar gum. Crosslinked PVP(CROSPOVIDONE), crosslinked CMC (Ac-Di-Sol), carboxymethylstarch-Na (PIRIMOJEL and EXPLOTAB), Pharmaburst and hydroxypropylcellulose (L HPC LH-11) are exemplary disintegrants.

A matrix can include, for example, Methocel K100 Prem-M or Eudragit RS PO powder, methacrylic copolymers (for example, methacrylic copolymer type A, methacrylic copolymer type B, Carbopol), and others known in the art.

Examples of glidants include colloidal silica, such as colloidal silicon dioxide, e.g., fumed silica (Cabosil, Aerosil), magnesium (Mg) trisilicate, powdered cellulose, starch, talc and tribasic calcium phosphate or combinations of these with fillers or binders, e.g., silicified microcrystalline cellulose (PROSOLV). Cabosil can also function as a flow enhancer/moisture protecting agent.

Further, fillers or diluents can include confectioner's sugar, compressible sugar, dextrates, dextrin, dextrose, lactose, mannitol, microcrystalline cellulose, for example microcrystalline cellulose having a density of about 0.45 g/cm³, such as AVICEL, powdered cellulose, sorbitol, sucrose and talc.

Lubricants include stearic acid and salts thereof, such as magnesium stearate, aluminum stearate, and calcium stearate, PEG 4000 to PEG8000, talc, hydrogenated castor oil, glycerol esters, Na-stearylfumarate, hydrogenated cotton seed oil and others. A common lubricant are stearic acid and Mg stearate.

Tablets and capsules can additionally be prepared with enteric coatings and other release-controlling coatings for the purpose of light protection, and swallowability. Examples of enteric coatings may include compounds prepared from, for example, methacrylic acid copolymers, cellulose acetate (and its succinate and phthalate version), styrol maleic acid co-polymers, polymethacrylic acid/acrylic acid copolymer, hydroxypropyl methyl cellulose phthalate, polyvinyl acetate phthalate, hydroxyethyl ethyl cellulose phthalate, hydroxypropyl methyl cellulose acetate succinate, cellulose acetate tetrahydrophtalate, acrylic resin, timellitate, and shellac. Exemplary polymers for enteric coatings include methacrylic copolymers such as Eudragit. Other suitable polymers for enteric coatings are known in the art. The coating may be colored with a pharmaceutically accepted dye. The amount of dye and other excipients in the coating liquid may vary and will not impact the performance of the immediate or extended release tablets. The coating liquid generally comprises film forming polymers such as hydroxy-propyl cellulose, hydroxypropylmethyl cellulose, cellulose ester or ether, an acrylic polymer or a mixture of polymers. The coating solution is generally an aqueous solution further comprising propylene glycol, sorbitan mono-oleate, sorbic acid, fillers such as titanium dioxide, a pharmaceutically acceptable dye.

Solid stable oral dosage forms for uses according to the present invention comprise a therapeutically effective amount of clavulanate as an active agent, and a filler as an additive. Further additives can include, but are not limited to, binders, disintegrants, lubricants, glidants, stabilizing agents, diluents, surfactants, film formers, pigments, softeners and antitacking agents and the like.

Potassium clavulanate has relatively low moisture content (<1% on a dry weight basis) when exposed to about 35% of relative humidity for 96 hr as shown in Table 10. However, it appears that deliquescence would eventually occur at any humidity above 40% relative humidity. Moisture absorption by dry potassium clavulanate exposed to 50% relative humidity occurs at a rate of approximately 1.44% per hour.

The use of lyophilization, or freeze drying, during the preparation of pharmaceutical compositions containing clavulanate increases the stability of the clavulanate tablet to about 97% (See Table 11).

Stable solid oral pharmaceutical compositions for uses according to the present invention can include clavulanate as the pharmaceutically active ingredient (API) at doses ranging from about 10 μg to 10 mg, for example, from about 0.1 mg to about 5 mg. In an exemplary embodiment, the clavulanate is a clavulanate salt, for example potassium clavulanate. It has been reported that clavulanic acid can alter CNS activity and behavior at i.p. doses ranging from 10 ng to 10 μg/kg (See U.S. Pat. No. 6,489,319).

According to the present invention, clavulanate can be administered in a number of dosage forms including, for example, immediate release and extended release dosage forms that contain from about 10 μg to about 10 mg clavulanate, for example from about 0.1 mg to about 5 mg clavulanate. Such dosage forms can be used for neuroprotection and the treatment of neurodegenerative disorders and symptoms thereof.

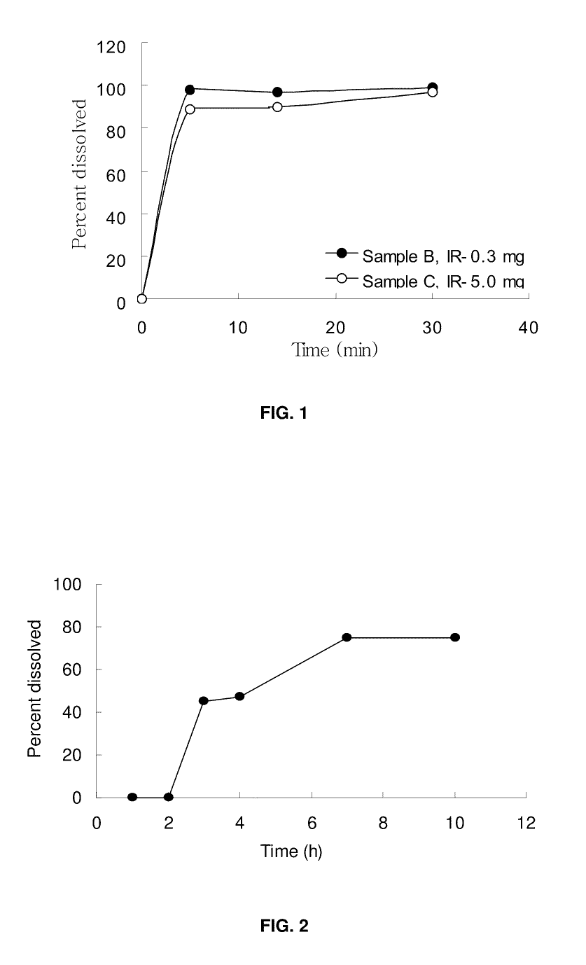

Immediate release forms desirably provide at least about 80% (w/v) dissolution of the clavulanate in less than about 30 minutes as determined by standard assays disclosed herein. The immediate release pharmaceutical compositions according to embodiments of the invention can be rapidly dissolved in an appropriate aqueous solution (e.g., water, saline, juice) or colloidal suspension (e.g., baby formula or milk) for convenient administration to patients unable to handle solid dosage forms. Illustrative of such patients are infants, children, and adults who may experience swallowing difficulties. In exemplary embodiments, at least about 80% of the clavulanate is dissolved in aqueous solution by about 15 minutes from the time that the composition is placed in the aqueous solution. In other embodiments, at least about 90% of the clavulanate is released to the aqueous solution by about 30 minutes, or by about 15 minutes, after exposure of the composition to the aqueous solution. As shown in FIG. 1, exemplary immediate release compositions useful in practicing the present invention release 90% of the clavulanate within 15 minutes after exposure to an aqueous solution.

Extended release compositions can release the active ingredient, i.e. clavulanate, over a long period, for example over about 8 hours or over about 10 hours. An extended release formulation can begin releasing the active ingredient as soon as the formulation reaches gastrointestinal track and continue to dissolve slowly and release the active ingredient in an approximately constant manner. This profile is desired because it provides steadier levels of the active ingredient in the bloodstream after administration. As shown in FIG. 2, exemplary extended release compositions useful in practicing the present invention can provide a substantially level release of the clavulanate up to about 8 to 10 hours after administration.

Pharmaceutical compositions for use according to embodiments of the invention provide important advantages. Control of water content is a major issue in the formulation and storage of clavulanate containing compositions because clavulanate is hygroscopic and is unstable or hydrolyzed in water. Use of lyophilization to prepare a stable immediate release or extended release composition provides unexpectedly enhanced stability, particularly when the clavulanate is combined with excipients prior to lyophilization.

Embodiments for use with the present invention can be a freeze dried composition of clavulanate can be used that includes: (1) forming a clavulanate composition by mixing clavulanate with at least one excipient; (2) freezing a quantity of the clavulanate composition, e.g., clavulanate, at 0° C. or below until converted into a frozen solid; and (3) dehydrating the clavulanate composition in an airtight container. The dehydrated (lyophilized) composition, including the drug, in powdered form can be mixed with other excipients before being compressed into tablets or prepared as sized beads.

The moisture content of the final dry formulation is low. The various embodiments used herein can have a final moisture content not exceeding about 10% (by weight), not exceeding about 5%, or not exceeding about 4%, or even lower. Dry formulations according to such embodiments of the invention are highly storage stable for extended periods, such as, for example, stable for about 30 days, about 60 days or about 90 days at conditions such as 25° C. and 60% relative humidity or 30° C. and 65% relative humidity. Upon dilution with the appropriate liquid, they are fully potent at substantially their stated initial dosage.

Formulations for use with the present invention can be prepared by dry blending a polymer, for example a matrix such as Eudragit (anionic copolymers of methacrylic acid and ethyl acrylate), a binder/diluent such as Maltrin M50 and/or a disintegrating agent such as Pharmaburst, filler, clavulanate, and other excipients (see examples), followed by granulating the mixture using water until proper granulation is obtained. The granulation is done by methods known in the art. The wet granules are freeze dried in a freeze dryer, sifted and ground to appropriate size. Lubricating agents can be mixed with the dried granulation to obtain the final formulation. As clavulanate is hygroscopic and labile in water, it is necessary to minimize the time mixture remains wet, for example, the processing time from weighing and granulation to freeze drying can be about 1 hr.

Compositions for use with the invention are administered orally, for example in the form of tablets or capsules. The tablets can be prepared by techniques known in the art and contain a therapeutically useful amount of clavulanate and such excipients as necessary to form the tablet by such techniques. Placebo particles can also prepared without clavulanate but with same composition.

Exemplary dosages of a stable solid clavulanate formulation that can be used for neuroprotection or treatment of neurodegenerative disorders in an adult human subject can be from about 5 mg per day to about 100 mg per day. In exemplary embodiments, the daily dosage is from about 5 mg to about 70 mg, for example from about 5 mg to about 50 mg or from about 7 mg to about 70 mg. Other exemplary dosages can be from about 10 mg per day to about 50 mg per day, about 5 mg per day, 7 mg per day, 10 mg per day, 20 mg per day, 25 mg per day, or 35 mg per day. As previously disclosed, the daily dosage can be administered once daily, twice daily, three times daily or more. As appreciated, administering fewer doses per day can generally require use of an extended release formulation. By way of example, a 10 mg daily dose can be administered as a single 10 mg dosage, two 5 mg doses, three doses of about 3.33 mg or four doses of 2.5 mg. Other dosage amounts can be calculated for a necessary dosage to a particular individual,

Stable solid dosage forms for administration according to the present invention can be provided as unit dosage forms. A unit dosage form is a single dose containing a predetermined amount of clavulanate active material. Examples of unit dosage forms include, without limitation, tablets, lozenges, capsules, and a packet containing a powder. Unit dosage forms for administration according to the present invention can include, for example, 0.1 mg, 0.25 mg, 1 mg, 1.5 mg, 2.0 mg, 2.5 mg, 5.0 mg, 7.5 mg, 10 mg or other amounts of clavulanate. A single dose can comprise a single unit dosage form, multiple unit dosage forms or partial unit dosage forms. By way of example, a 5 mg dose can be administered as two unit dosage form each containing of 2.5 mg clavulanate, a single unit dosage form containing 5.0 mg clavulanate, or half of a unit dosage form containing 10 mg clavulanate. Other dosage amounts comprising other unit dosage forms can be readily calculated.

Pharmacokinetic Study

The bioavailability study for the formulations of the invention was measured by administering the immediate or extended formulation in a tablet form to healthy subjects and measuring the levels of clavulanate in the plasma at different time intervals over a period of twenty four hours. Plasma samples were assayed for clavulanate by BAS Analytics (West Lafayette, Ind.) using a validated high performance liquid chromatographic procedure similar to that described in the literature. See for example, Chu S-Y, et al., “Simultaneous determination of clarithromycin and 14(R)-hydroxyclarithromycin in plasma and urine using high performance liquid chromatography with electrochemical detection”, J. Chromatography, 571, pp 199-208 (1991).

### EXAMPLES

The following examples are for purpose of illustration only and are not intended to limit the scope of the appended claims.

### Example 1

### Preparation of Clavulanate Tablets

### Example 1A

### Preparation of Immediate Release Clavulanate Tablet using Potassium Clavulanate Powder

Exemplary description of tablet preparation process: A wet granulation tablet formulation process has been discovered where water is included in a granulation step, followed by drying to obtain granules of low water content (<3%). The dried formulation is non-hygroscopic compared with prior art formulations, but maintains equivalent physical characteristics (for example, dissolution, disintegration, bioavailability and other physical properties) of the tablet prepared therefrom. The tablet preparation was carried out by granulating the clavulanate with water in the presence of binder/diluent.

For the preparation of sample C, Maltrin M150 (130 g) was dissolved in purified water and potassium clavulanate (API; 59.5 g) was added. Prosolve SMCC-50 (490.5 g), Pharmaburst (130.0 g), L HPC LH-11 (120.0 g), Acdisol (20.0 g) and stearate acid (50 g) were weighed and mixed in a bag by shaking and rotating the bag. The mixture was transferred to the bowl of a Hobart mixer and the API/Maltrin M150 solution was added to the mixture with stifling for 10 minutes. After wet massing was completed, the contents of the bowl of the Hobart mixer were transferred into an extruder and extruded. The extrudate was placed into the spheronizer and the spheronized material was collected in a bag and lyophilized in a gortex-lyoguard tray. The dried material was screened and compressed into tablets or prepared into sized beads. Sample A and B were prepared in the same way as sample C.

### Example 1B

### Preparation of Immediate Release Clavulanate Tablet using Clavitesse™

For the preparation of sample D, Clavitesse™ (API; 50.6 g), Prosolve SMCC 50 (213.4 g), Pharmaburst (100.0 g), Acdisol (8.0 g), Cabosil (8.0 g) and magnesium stearate (20.0 g) were weighed and lyophilized overnight in a gortex-lyoguard tray at 2-8° C. On the next day, the API, Prosolve SMCC 50, Pharmaburst and Acdisol were mixed in a bag, screened through # 40 mesh, unloaded into a V blender and mixed for 7 minutes. The mixture was screened again and mixed in the V blender for 4 min. The Cabosil and magnesium stearate were screened and mixed with the mixture containing API in the V blender for 4 min. The blend was lyophilized overnight in a gortex-lyoguard tray. The material was compressed into tablets and tablets were lyophilized in the gortex-lyoguard tray and packaged. Sample E was prepared in the same way as sample D.

### Example 1C

### Preparation of Extended Release Clavulanate Tablet using Clavitesse™

For the preparation of sample F, suitable amounts of Clavitesse (API; 41.07 g), Methocel K100LV Prem CR (90.0 g), Isomalt (83.55 g), Avicel PH-112 (80.04 g), Cabosil (1.5 g), Talc (2.4 g) and magnesium stearate (1.5 g) were weighed and dried in Freeze dryer overnight with application in a gortex-lyoguard tray at 2-8° C. Each ingredient was screened and collected in a separate bag. API and Methocel K100LV Prem CR were loaded into a V blender, mixed, screened through a suitable sieve and mixing was continued. Avicel PH-112 and Isomalt were added to the mixture and mixed. The resulting mixture was screened and mixed again. Cabosil and Talc were mixed and added into the mixture and mixed. Magnesium stearate was mixed with the mixture in the V blender. The final blend was freeze dried overnight in a gortex-lyoguard tray and compressed into tablets or prepared into sized beads. Tablets were compressed at higher hardness for extended release coating. Tablets or beads were coated with delay release polymer, Eudragit.

### Example 1D

### Preparation of Extended Release Clavulanate Tablet using Potassium Clavulanate Powder

For the preparation of extended release tablet using potassium clavulanate, Sample G, potassium clavulanate (API; 20.69 g) was screened through # 60 mesh and other excipients, Methocel K100LV Prem CR (90.02 g), Isomalt (83.56 g), Avicel PH-112 (100.41 g), Cabosil (1.52 g), Talc (2.4 g) and magnesium stearate (1.5 g), were screened through # 40 mesh. Each ingredient was collected in a separate bag. The API and Methocel K100LV Prem CR were loaded into a V blender and mixed for 5 minutes. The mixture was screened and mixed for 5 additional minutes. The Avicel PH-112 and Isomalt were added to the mixture and mixed in the V blender for 5 minutes. The resulting mixture was screened and mixed for 5 additional minutes. The Cabosil and Talc were mixed and loaded into the mixture and then the resulting mixture was mixed for 2 minutes. Finally, magnesium stearate was mixed with the mixture in the V blender for 3 minutes and the final blend was lyophilized overnight in the gortex-lyoguard tray and then compressed into tablets or prepared into sized beads. Tablets were compressed at higher hardness for extended release coating. Tablets or beads were coated with delay release polymer, Eudragit. Sample H and I were prepared in the same way with sample G.

### Example 2

### Assay of Clavulanate

The clavulanate content of the prepared pharmaceutical composition was measured by Waters HPLC (high performance liquid chromatography) system (column: μBondapack-NH₂ (10 μm) 300 mm×3.9 mm, Mobile phase: CH₃CN:pH 5.2 KH₂PO₄=65:35, Flow rate: 1.0 ml/min) using the following procedure: About 10 tablets were accurately weighed and grinded, 100 ml of water added and the mixture sonicated for 20 min. After dilution with water, a portion of solution was filtered and injected into HPLC. The major peak was identified by the retention time of the sample that corresponded to the chromatogram of the standard preparation by HPLC. The % clavulanate was calculated based on analyte response factor compared to the response factor of the reference standard.

Linearity of clavulanate standard curve was verified at 25, 50, 75, 100, 125, 150% of reference standard at nominal concentration of 0.01 mg/ml. R² was 0.9998. At nominal concentration of 0.01 mg/ml of clavulanate, precision was verified using six samples with percent of RSD 1.4. Accuracy was determined by preparing, in triplicate, and analyzing spiked placebo blends at 50%, 100%, and 150% of 0.01 mg/ml.

### Example 3

### Exemplary Formulation and Characteristics

The following experiments describe tablet formulation designed as immediate release (IR) tablet and extended release (ER) tablet with different doses. The following table also represents the physical properties of tablets according to the present formulation.

### Example 3A

### Immediate Release Composition Using Potassium Clavulanate

Immediate release compositions were prepared from potassium clavulanate powder and excipients as shown in Table 1 using the method described above.

TABLE 1

| | | Sample A, | Sample B, | Sample C, |
|---|---|---|---|---|
| Ingredient (mg) | Function | 0.1 mg/tablet | 0.3 mg/tablet | 5 mg/tablet |
| Potassium Clavulanate | API* | 0.1 | 0.357 | 5.95 |
| Maltrin M150 | Binder/diluent | 15 | 15 | 13 |
| Prosolve SMCC 50 | Filler | 50 | 50 | 49.05 |
| Pharmaburst | Disintegrating agent | 15 | 15 | 13 |
| L HPC LH-11 | Disintegrating agent | 15 | 15 | 12 |
| Acdisol | Disintegrating agent | 0.1 | 0.1 | 2 |
| Stearic acid | Lubricant | 4.8 | 4.543 | 5 |

API*: Active pharmaceutical ingredient.

Table 2 summarizes the characteristics of immediate release tablet using potassium clavulanate powder. Sample C tablet showed excellent stability, containing 94.4% of potassium clavulanate after 1 week at 2-8° C.

TABLE 2

| | | Sample A, | Sample B, | Sample C, |
|---|---|---|---|---|
| Parameter | Unit | 0.1 mg/tablet | 0.3 mg/tablet | 5 mg/tablet |
| Weight | mg | 106 | 106 | 101 |
| Hardness | KP | 5 | 5 | 3-5 |
| Thickness | mm | 0.155 | 0.155 | 3.6-3.8 |
| Disintegration Time | sec | 15 | 15 | 20 |
| Assay | % | 95.3 | 95.3 | 89.4-92.9% |
| 1 Week Assay 2-8° C. | % | — | — | 94.4 |
| Content Uniformity | RSD | 2.5 | 2.6 | 1 |
| Dissolution | % dissolved | — | 98% in 5 min | 89% in 5 min |
| Moisture Content-Final | % | — | 0.91 | 3.14 |

### Example 3B

### Immediate Release Composition Using Clavitesse™

Immediate release compositions comprising 5 mg of clavulanate were prepared using Clavitesse™ as shown in Table 3.

TABLE 3

| | | Sample D, | Sample E, |
|---|---|---|---|
| Ingredient (mg) | Function | 5 mg/tablet | 5 mg/tablet |
| 1:1 mixture of potassium | API* | 12.65 | — |
| clavulanate and micro- | | | |
| crystalline cellulose | | | |
| 1:1 mixture of | API* | — | 12.62 |
| potassium clavulanate | | | |
| and silicon dioxide | | | |
| Prosolve SMCC 50 | Filler | 53.35 | 53.38 |
| Pharmaburst | Disintegrating | 25 | 25 |
| | agent | | |
| Acdisol | Disintegrating | 2 | 2 |
| | agent | | |
| Cabosil | Flow enhancer/ | 2 | 2 |
| | moisture | | |
| | protectant | | |
| Magnesium stearate | Lubricant | 5 | 5 |

API*: Active pharmaceutical ingredient.

Table 4 summarizes the characteristics of immediate release tablet using Clavitesse™

TABLE 4

| | | Sample D, | Sample E, |
|---|---|---|---|
| Parameter | Unit | 5 mg/tablet | 5 mg/tablet |
| Weight | mg | 103-104 | 108 |
| Hardness | KP | 5-7 | 5-7 |
| Disintegration Time | min | <1 min | <2 min |
| Moisture content | % | 3.24 | 3.40 |

### Example 3C

### Extended Release Composition Using Clavitesse™ and Potassium Clavulanate Powder

Extended release compositions were prepared using Clavitesse™ or potassium clavulanate powder as shown in Tables 5-8.

TABLE 5

| | | Sample F, | Sample G, | Sample H, | Sample I, |
|---|---|---|---|---|---|
| Ingredient (mg) | Function | 5 mg/tablet | 5 mg/tablet | 0.3 mg/tablet | 1.0 mg/tablet |
| 1:1 Mixture of potassium | API* | 13.69 | — | | |
| clavulanate and | | | | | |
| microcrystalline cellulose | | | | | |
| Potassium clavulanate | API* | — | 6.894 | 0.357 | 1.19 |
| Klucel LF | Matrix | | | 6 | — |
| (Hydroxypropylcellulose) | | | | | |
| Methocel K100 Prem-M | Matrix | | | — | 37 |
| Eudragit RS PO powder | Matrix | | | 20 | — |
| Methocel K100LV Prem | Matrix | 30.0 | 30.0 | | |
| CR | | | | | |
| Anhydrous lactose | Filler | | | 30 | — |
| Avicel PH-112 | Filler | 26.67 | 27.85 | 41.24 | — |
| Avicel PH-113 | Filler | | | — | 20 |
| Isomalt | Filler | 27.85 | 33.47 | — | 40 |
| Cabosil | Glidant | 0.5 | 0.5 | 0.8 | 0.5 |
| Magnesium stearate | Lubricant | 0.5 | 0.5 | 1.6 | 0.5 |

| | | | | | |
|---|---|---|---|---|---|
| Talc | Lubricant | 0.8 | 0.8 | — | 0.8 |

| | | | | | | | | |
|---|---|---|---|---|---|---|---|---|
| Total | 100 | mg | 100 | mg | 100 | mg | 100 | mg |

API*: Active pharmaceutical ingredient.

TABLE 6

| | | Sample J, | Sample K, |
|---|---|---|---|
| Ingredient | Function | 5 mg/tablet | 5 mg/tablet |
| 1:1 Mixture of potassium | API* | 13.69 | — |
| clavulanate and micro- | | | |
| crystalline cellulose | | | |
| Potassium clavulanate | API* | — | 6.894 |
| Eudragit S100 | Matrix | 25.0 | 25.0 |
| Avicel PH-112 | Filler | 26.67 | 27.85 |
| Isomalt | Filler | 30.34 | 35.96 |
| Ethocel 10 cps | Glidant | 1.5 | 1.5 |
| Acetyltributyl citrate | Lubricant | 2.0 | 2.0 |
| Talc | Lubricant | 0.8 | 0.8 |
| Total | | 100 mg | 100 mg |

API*: Active pharmaceutical ingredient.

TABLE 7

| | | Sample L, | Sample M, |
|---|---|---|---|
| Ingredient | Function | 5 mg/tablet | 5 mg/tablet |
| 1:1 Mixture of potassium | API* | 13.69 | — |
| clavulanate and micro- | | | |
| crystalline cellulose | | | |
| Potassium clavulanate | API* | — | 6.894 |
| Carbopol 971P | Matrix | 20.0 | 20.0 |
| Carbopol 974P | Matrix | 35.0 | 35.0 |
| Pharmatose DCL21 | Filler | 29.51 | 36.31 |
| Cabosil | Glidant | 0.5 | 0.5 |
| Magnesium stearate | Lubricant | 0.5 | 0.5 |
| Talc | Lubricant | 0.8 | 0.8 |
| Total | | 100 mg | 100 mg |

API*: Active pharmaceutical ingredient.

TABLE 8

| | | Sample N, | Sample O, |
|---|---|---|---|
| Ingredient | Function | 5 mg/tablet | 5 mg/tablet |
| 1:1 Mixture of potassium | API* | 13.69 | — |
| clavulanate and micro- | | | |
| crystalline cellulose | | | |
| Potassium clavulanate | API* | — | 6.894 |
| Methacrylate copolymer type A | Matrix | 30.0 | 30.0 |
| Methacrylate copolymer type B | Matrix | 25.0 | 25.0 |
| Avicel PH-112 | Filler | 29.51 | 36.3 |
| Cabosil | Glidant | 0.5 | 0.5 |
| Magnesium stearate | Lubricant | 0.5 | 0.5 |
| Talc | Lubricant | 0.8 | 0.8 |
| Total | | 100 mg | 100 mg |

API*: Active pharmaceutical ingredient.

Table 9 summarizes the characteristics of extended release tablet using Clavitesse™ and potassium clavulanate powder

TABLE 9

| | | | | | Sample I, |
|---|---|---|---|---|---|
| | | Sample F, | Sample G, | Sample H, | 1.0 mg/ |
| Parameter | Unit | 5 mg/tablet | 5 mg/tablet | 0.3 mg/tablet | tablet |
| Weight | mg | 99.9-102.4 | 92.0-108.3 | 104-105 | 108 |
| Hardness | KP | 9.9-14.0 | — | 7-9 | 10 |
| Assay | % | 105.9 | 96.2 | 0.756 | 3.44 |

### Example 4

### In Vitro Dissolution Studies

Tablets were placed in the 500 ml of solvent (deionized water for immediate release tablets; pH 1.2 solution for first 2 hrs and then pH 7.0 of citrate buffer for the next 8 hrs for extended release tablets). The mixture was swirled at 100 rpm and at 37° C. and a sample periodically collected and tested for the amount of dissolved clavulanate by HPLC.

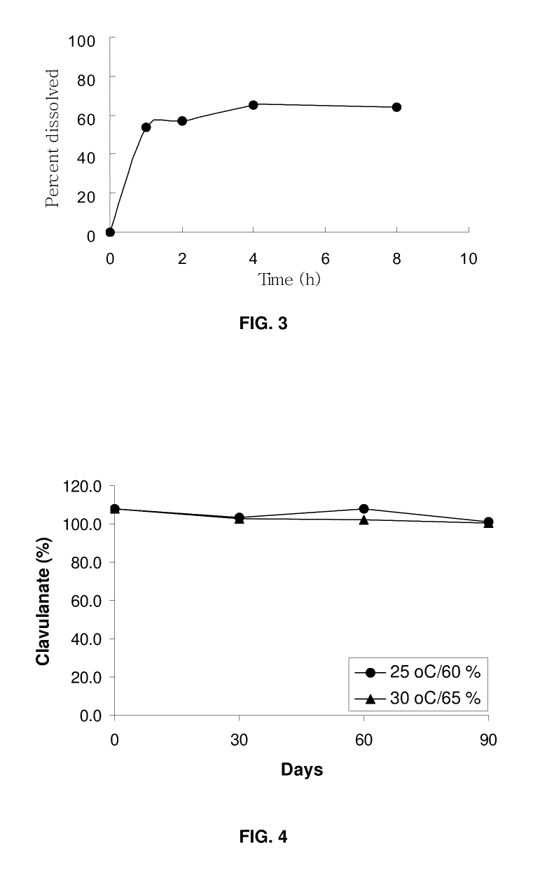

The results are shown in FIGS. 1-3. FIG. 1 is a graph showing the in vitro dissolution profiles of clavulanate immediate-release formulations of Sample B and Sample C. As shown in FIG. 1, 90% or more of clavulanate in the immediate release tablet was dissolved within 15 min after exposure to the aqueous solution. FIG. 2 is a graph showing the in vitro dissolution profile of the clavulanate extended-release formulation of Sample F. FIG. 3 is a graph showing the in vitro dissolution profile of the clavulanate extended-release formulation of Sample I. As shown in FIGS. 2 and 3, the total dose of clavulanate in the extended release tablet was slowly released via erosion and dissolution mechanisms over a period of at least about 8 to 10 hours. Release of clavulanate in the extended release form was not detected in pH 1.2 solution.

### Example 5

### Stability Test

Potassium clavulanate in its solid form is both hygroscopic and unstable in the presence of water vapor. A stability study of clavulanate was conducted with monitoring by chromatographic methods. The static or equilibrium approach was approached by storing samples in chambers at different relative humidity in an attempt to generate a sorption isotherm. The sorption isotherm represents the quantitative relationship between the equilibrium moisture content and relative humidity (RH) in the atmosphere. Table 10 shows the change of the water content in potassium clavulanate powder after exposed to the different humidity conditions.

TABLE 10

| | | Moisture Content (%) | Moisture Content (%) |
|---|---|---|---|
| Time | % RH | (g H₂O/g wet solid) | (g H₂O/g dry solid) |
| 96 hr | 33 | 0.708 | 0.713 |
| | 35 | 0.733 | 0.737 |
| | 37 | 0.842 | 0.848 |
| | 39 | 1.264 | 1.280 |
| | 41 | 1.542 | 1.566 |
| | 43 | 3.976 | 4.140 |
| | 45 | 4.778 | 5.018 |
| | 47 | 12.823 | 14.708 |

As shown in Table 10, potassium clavulanate has relatively low moisture content (<1% on a dry weight basis) when exposed to about 35% or less of relative humidity for 96 hr. However, it appears that deliquescence would eventually occur at any humidity above about 40% relative humidity. Moisture absorption by dry potassium clavulanate exposed to about 50% relative humidity occurs at a rate of approximately 1.44% per hour.

Potassium clavulanate is an exceptionally difficult material to formulate, being extremely moisture and heat sensitive. Degradation readily occurs in the presence of water and aqueous media. Several methods were tested to find a suitable condition for removing moisture after wet granulation that keeps the active ingredient clavulanate intact. The material in sample C was prepared by wet granulation and spheronized. The moisture containing spheronized formulation was transferred to trays and subjected to different storage conditions for the removal of moisture.

As summarized in Table 11, storage at 30° C. for 69 hr (storage 1), or storage at 45° C. for 75 hr (storage 2), resulted in the degradation of potassium clavulanate up to 45% and 60% respectively. Drying in a fluid bed system resulted in degradation of the clavulanate by 13% in only 1.5 hr. These data suggest that potassium clavulanate is also temperature sensitive. Lyophilization retained 97% of the active ingredient after 21 hrs of the freeze drying process. The results in Table 11 show that lyophilization of clavulanate can be used to reduce the content of moisture in a clavulanate formulation and increase the stability of the formulation.

TABLE 11

| Method | Temp (° C.) | Time (hr) | Clavulanate (%) |
|---|---|---|---|
| Storage 1 | 30 | 69 | 55 |
| Storage 2 | 45 | 75 | 40 |
| Fluid bed | 40 | 1.5 | 87 |
| Freeze dry | Sub-zero | 21 | 97 |

Stability of immediate release tablets prepared from Clavitesse™, Sample D and

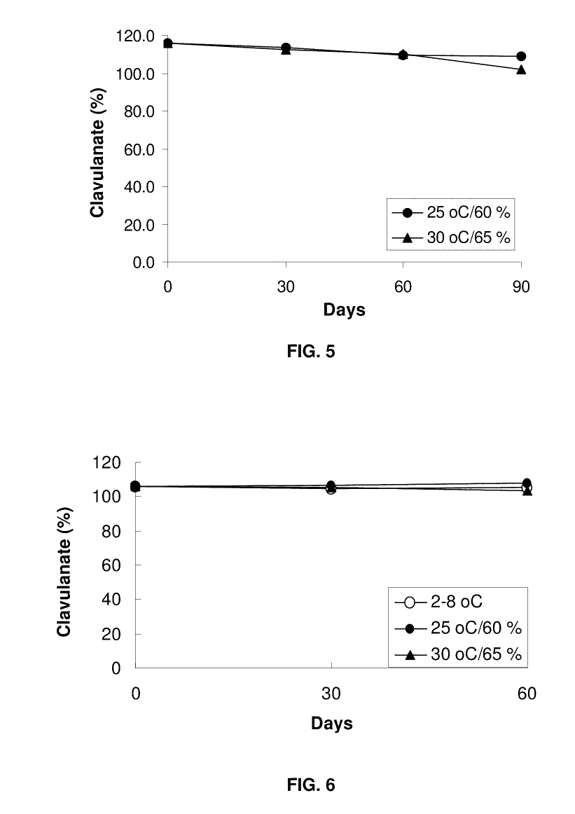

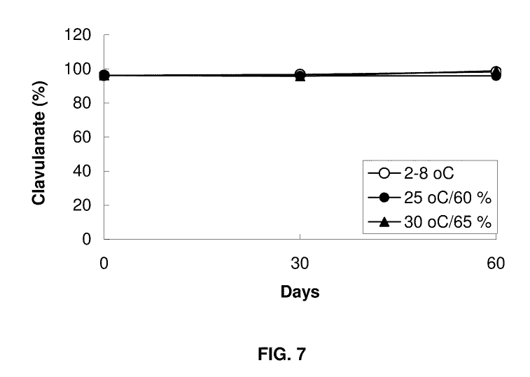

Sample E, was evaluated for up to 3 months. FIG. 4 is a graph showing the stability of Sample D (5 mg/tablet of 1:1 mixture of potassium clavulanate and microcrystalline cellulose) at 25° C./60% humidity and 30° C./65% humidity. FIG. 5 is a graph showing the stability of Sample E (5 mg/tablet of 1:1 mixture of potassium clavulanate and silicon dioxide) at 25° C./60% humidity and 30° C./65% humidity. As shown in Table 4 and in FIGS. 4 and 5, both tablets prepared according to Samples D and Sample E initially contained less than 4%-moisture and were degraded less than 7% at 25° C./60% humidity, a relative high humidity condition for clavulanate. Stability of extended release tablets prepared from Clavitesse™, Samples F and G were evaluated for up to 2 months. FIG. 6 is a graph showing the stability of Sample F (5 mg/tablet of 1:1 mixture of potassium clavulanate and microcrystalline cellulose) at 2-8° C., 25° C./60% humidity and 30° C./65% humidity. FIG. 7 is a graph of the stability of Sample G (5 mg/tablet) at 2-8° C., 25° C./60% humidity and 30° C./65% humidity. As shown in Table 5 and in FIGS. 6 and 7, the tablets prepared according to Samples F and G initially contained less than 4%-moisture and were degraded less than 1.6% at 30° C./65% humidity, a relative high humidity condition for clavulanate. Therefore it appears that microcrystalline cellulose or silicon dioxide in Clavitesse™ may further contribute the increase of stability of potassium clavulanate by capturing the moisture in a tablet.

### Example 6

### Pharmacokinetic Study

The amount of clavulanate in the plasma of beagle dogs was measured by LC/MS/MS method. The chromatographic separation of the analytes was performed on a reverse-phase PLRP-S polymeric column. The retention time of potassium clavulanate and tazobactam (reference compound) were 8.51 and 8.54 min, respectively. The overall chromatographic run time was 25 min. The M/S analysis was performed on an Applied Biosystems' API 2000 triple-quardrupole mass spectrometer by multiple reaction monitoring in negative electrospray ionization mode. The mass spectral data were analyzed by Analyst 1.4.1 (Applied Biosystems). The pharmacokinetic analysis was conducted by using PK Solutions 2.0 (Summit Research Services).

### Example 6A

### Oral Administration of Immediate Release (IR) Tablet in Male Beagle Dogs

Three male Beagle dogs were used throughout the study in a cross-over design with washout period between treatments. The dogs were given the test substances as IR tablet of Example 3A via oral routes with no shorter than 24 hr washout period between dosing. The animals were fasted overnight before the administration of the test substance and fed 4 hr post-dosing. During all the treatments, blood samples (1.5 ml) were withdrawn from the cephalic vein by venipuncture into heparinized tubes at 0, 5, 15, 30 min, 1, 1.5, 2, 2.5, 3, 4, 6, 9 and 12 hr after dosing. Plasma was obtained via centrifugation at 3,000 rpm for 10 min and analyzed by an LC-MS/MS system. The associated mean pharmacokinetic parameters are provided in Table 12.

### Example 6B

### IV Administration of Potassium Clavulanate Solution in Male Beagle Dogs

Three male beagle dogs were used throughout the study in a cross-over design with washout period between treatments. The dogs were given the test substances as aqueous solution via intravenous routes with no shorter than 24 hr washout period between dosing. The animals were fasted overnight before the administration of the test substance and fed 4 hr post-dosing. During all the treatments, blood samples (1.5 ml) were withdrawn from the cephalic vein by venipuncture into heparinized tubes at 0, 5, 15, 30 min, 1, 1.5, 2, 2.5, 3, 4, 6, 9 and 12 hr after dosing. Plasma was obtained via centrifugation at 3,000 rpm for 10 min and analyzed by an LC-MS/MS system. The associated mean pharmacokinetic parameters are provided in Table 12.

### Example 6C

### Oral Administration of Extended Release (ER) Tablet in Male Beagle Dogs

Four male beagle dogs were used throughout the study in a cross-over design with washout period between treatments. The dogs were given the test substances as ER tablet of Example 3C via oral routes with no shorter than 24 hr washout period between dosing. The animals were fasted overnight before the administration of the test substance and fed 4 hr post-dosing. During all the treatments, blood samples (1.5 ml) were withdrawn from the cephalic vein by venipuncture into heparinized tubes at 1, 2, 3, 4, 5, 6, 7, 8, 9, 10, 11 and 12 hr after dosing. Plasma was obtained via centrifugation at 3,000 rpm for 10 min and analyzed by an LC-MS/MS system. The associated mean pharmacokinetic parameters are provided in Table 12.

TABLE 12

| | | | | | | | | Oral | |
|---|---|---|---|---|---|---|---|---|---|
| | | IV | | Oral (IR tablet) | | | | (ER tablet) | |
| PK Parameter* | | mean | SD | mean | SD | mean | SD | mean | SD |
| Dose | (mg) | 4.2 | — | 3.5 | — | 7.4 | — | 21.6 | — |
| Tₘₐₓ | (hr) | — | — | 1.2 | 0.3 | 1.2 | 0.3 | 2.8 | 1.0 |
| Cₘₐₓ | (ng/ml) | — | — | 125.8 | 80.0 | 413.7 | 127.9 | 821.3 | 492.7 |
| AUC0-t | (hr · ng/ml) | 684.4 | 74.6 | 175.6 | 101.8 | 498.4 | 70.8 | 1702.4 | 580.6 |
| CL | (l/hr) | 5.8 | 0.7 | | | | | | |
| Vd | (l) | 4.4 | 0.5 | — | — | — | — | — | — |
| Vss | (l) | 3.8 | 0.4 | — | — | — | — | — | — |
| t_{1/2} | (hr) | 0.52 | 0.02 | 0.49 | 0.09 | 0.46 | 0.02 | 1.9 | 1.5 |
| MRT_{inf} | (hr) | 0.65 | 0.01 | 1.6 | 0.1 | 1.7 | 0.3 | 3.4 | 1.5 |
| F | (%) | 100 | — | 29.9 | 14.7 | 41.4 | 4.7 | 45.4 | 15.5 |

*PK parameters:
Tₘₐₓ: time to maximum concentration,
Cₘₐₓ: maximal concentration,
AUC: area under the curve,
CL: clearance,
Vd: volume of distribution,
Vss: volume of distribution at steady state,
t_{1/2}: half-life,
MRT_{inf}: mean residence time,
F: bioavailability

Potassium clavulanate was shown to be well absorbed in fasted animals, with an average bioavailability of 30˜41%, when given orally. The apparent terminal half-life was 0.5 hr.

### Example 7

### Parkinson's Disease Animal Model

Procedure

The neuroprotectant effects of clavulanate was tested in an MPTP (1-methyl-4-phenyl-1,2,3,6-tetrahydropyridine) induced Parkinson's disease animal model. Eight week old male C57BL/6 mice were separated into six groups of ten. Three times (one time per day) prior to MPTP treatment, ten animals were treated with clavulanate at a particular dose and route of administration (Groups 2-5) while the remaining animals (groups 1 and 6) were given saline vehicle. (See Table 13.) MPTP was given four times intraperitoneally at a dose of 20 mg/kg (total of 80 mg/kg). Two times (one time per day) post MPTP treatment, animals were given another administration of clavulanate or saline vehicle. Animals were tested for changes in behavior. Animals that survived MPTP treatment were sacrificed 7 days later and their brains examined for histological changes in the substantia nigra pars compacta (SNpc). Ten untreated control animals of the same weight and age as the experimental animals were sacrificed and their hippocampal morphology used as a standard for comparison.

TABLE 13

| Group | MPTP | Clavulanate Dose | | Route of Administration |
|---|---|---|---|---|
| Group 1 | Yes | None (saline only) | | |
| Group 2 | Yes | 0.01 | mg/kg | Interperitoneal |
| Group 3 | Yes | 0.1 | mg/kg | Interperitoneal |
| Group 4 | Yes | 0.1 | mg/kg | Gavage |
| Group 5 | Yes | 1.0 | mg/kg | Gavage |

| | | |
|---|---|---|
| Group 6 | No | None (saline only) |

Behavior Testing

The pole test has been effectively used to assess rodent models of Parkinson's disease.

In this test a mouse is placed atop a metal pole facing upward, and the time to orient down and descend is measured. Motor impairment correlates with an increased time to orient down and descend.

Mice were placed atop a pole (50-cm high and 1-cm wide) that had been wrapped in wire. The base of the pole was placed in the animal's home cage. The time required to orient downward and descend the pole was then recorded. MPTP-treated mice are known to have slower times orienting down and descending the pole. On the test day, the animals were recorded over five trials, and the average over the five performances was calculated. If the mouse fell off the pole or was unable to climb down the pole in any given trial, the longest time from among that animal's previous trials was recorded for the unsuccessful run.

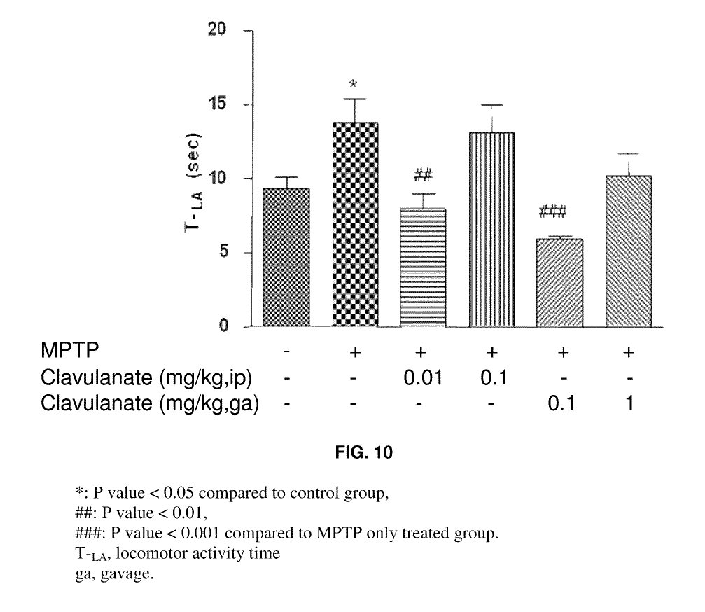

The MPTP-saline group showed significant increase of locomotor activity time compared to the normal group. The locomotor activity time of TH-IR (tyrosine hydroxylase-immunoreactive) neurons was significantly decreased in low dose clavulanate treated group (0.01 mg/kg, i.p. and 0.1 mg/kg, ga). But, the time was not significantly different from MPTP and high dose clavulanate treated group. FIG. 10 illustrates the behavioral effects of clavulanate on MPTP-induced neurotoxicity using pole test in mouse PD model. Each column represents the Mean±S.E.M. *: P value<0.05 compared to control group, ##: P value<0.01, ###: P value<0.001 compared to MPTP only treated group. (T-_{LA}, locomotor activity time; ga, gavage.) Table 14 shows the behavioural effects of clavulanate on locomotor activity time of MPTP-induced PD model.

TABLE 14

| | | | MPTP + | MPTP + | MPTP + | MPTP + |
|---|---|---|---|---|---|---|
| | | | clavulanate | clavulanate | clavulanate | clavulanate |
| T-_{LA} | Normal | MPTP | 0.1 mg/kg ga | 1 mg/kg ga. | 0.01 mg/kg i.p. | 0.1 mg/kg i.p. |
| average | 9.361 | 13.784 | 5.898 | 10.213 | 7.981 | 13.086 |
| s.e.m | 0.733 | 1.566 | 0.203 | 1.489 | 0.983 | 1.866 |

Tissue Processing

On completion of the experiment, animals were anesthetized by IP injection of 10 mg/kg pentobarbital sodium, then perfused transcardially with 10 ml of PBS at pH 7.4, follow by 50 ml of 4% paraformaldehyde in PBS over a 5 min period. The brains were removed from the skull and post-fixed by immersion in the same fixative solution for 4 h, then transferred to 30% sucrose in PBS. After equilibration in the sucrose solution, coronal sections were cut using the cryocut at a thickness of 40 μm into storing solution and stored at 4° C. prior to staining.

Tyrosine Hydroxylase (TH) Immunohistochemistry

Immunohistochemistry was carried out on free floating sections. All stains were carried out on a 1 in 5 series of sections. All sections were stained simultaneously using the same solutions of antibodies and ensuring that incubation times and washes were the same for each brain. The following protocol was used. Sections were washed in PBS. Endogenous peroxidase enzyme activity was quenched using a 10 min immersion in 3% hydrogen peroxide in PBS, follow by washing and re-equilibration in PBS. After 1 h preincubation period in a solution of 3% normal goat serum/0.1% Trixon X-100 in PBS, sections were incubated in a polyclonal anti-TH (tyrosine hydroxylase) antibody (Chemicon) at a 1:2,000 dilution in 1% normal goat serum/0.1% Triton X-100 overnight at room temperature. After thorough washing, a biotinylated anti-rabbit antibody (Vector, 1:200) in 0.1% Triton X-100 in PBS was applied for 90 min. The sections were then washed for 15 min before application of ABC solution (Vector, 1:100) in PBS for 1 hr, followed by thorough washing in PBS. The horseradish peroxidase label was revealed by 3 min incubation in a 0.02% solution of DAB in PBS containing 0.1 μl/ml of hydrogen peroxide. Sections were mounted on gelatin-coated microscope slides dehydrated in an ascending series of alcohols, cleared and cover-slip using Histomount medium.

Quantitation of Data and Statistical Analysis

Neurons were counted using the optical fractionator, an unbiased method for cell counting that is not affected by either the volume of reference or the size of the counted elements (neurons). This method was carried out using a computer-assisted image analysis system, consisting of an Axiophot photomicroscope (Zeiss, Germany) comprising a Zeiss planapochromat objective equipped with a computer-controlled motorized stage, a video camera, and the Stereo Investigator software (MicroBrightField, Williston, Vt.). Cell counts were performed by counting the number of neurons on the SNpc of every fifth section throughout the entire extent of the SN using a standard mouse atlas (Paxinos and Franklin, 2004) as anatomical reference.

Statistical analysis for each experiment group were assessed by Students t test.

Differences were considered significant when p<0.05. All statistical analyses were performed using GraphPad Prism software.

Results

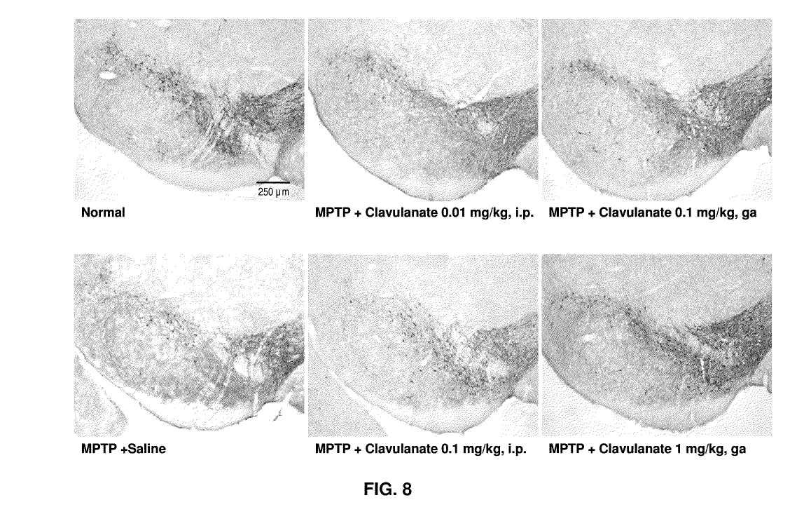

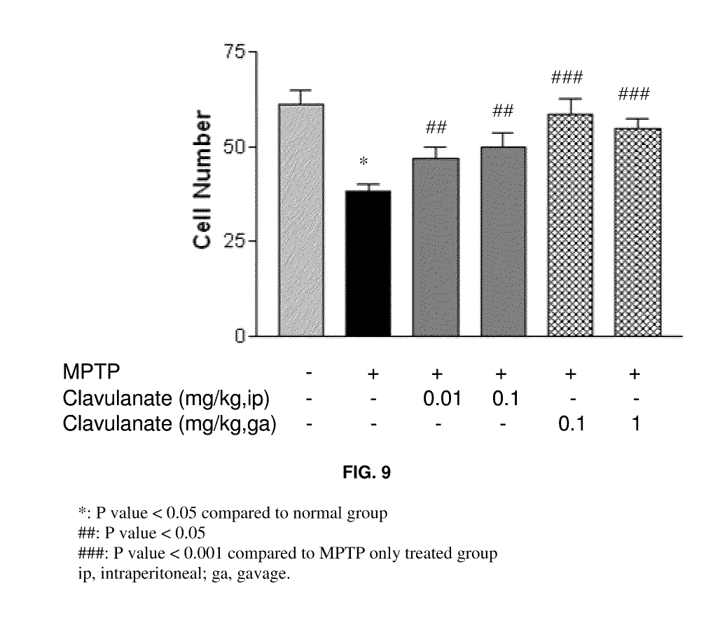

In the normal group, many TH-immunoreactive (IR) neurons were distributed in the substantia nigra pars compacta, and some TH-IR neurons were scattered in substantia nigra pars reticulata. MPTP-saline groups showed significant decrease of TH-IR neurons compared to normal group. In the clavulanate treated group (ip and ga), TH-IR neurons were significantly protected from MPTP-induced TH-IR neuronal damage. FIG. 8 illustrates the immunohistochemistry for tyrosine hydroxylase (TH) in the substantia nigra pars compacta (SNpc). The number of TH-positive neurons were significantly decreased in MPTP-saline group compared to normal group. The number of TH-positive neurons were well preserved in MPTP-clavulanate treatment group. FIG. 9 shows the effects of clavulanate treatment on substantia nigra pars compacta (SNpc) neuron survival in MPTP-treated animals. In MPTP-treated group there was a significant decrease in TH-positive neurons within the SNpc. In both clavulanate-treated groups (ip and ga) there was a significant protection of TH-positive neurons within the SNpc, with a greater protection of cells following gavage treatment. TH-positive SNpc neurons were bilaterally counted for at the widest dimension of the SNpc at AP-3.16 lateral to the roots of the third cranial nerve separating the medial and lateral SNpc. (*: P value<0.05 compared to normal group, ##: P value<0.05, ###: P value<0.001 compared to MPTP only treated group. ip, intraperitoneal; ga, gavage.)

### Example 8

### Kainate Animal Model

Procedure

As a neuroprotectant, clavulanate was tested in the Kainate animal model. Thirty male Sprague Dawley rats weighing 300-350 grams were separated into three groups. One hour prior to kainate treatment, seven animals are treated with clavulanate at an IP dose of 10 μg/kg while the remaining animals were given saline vehicle. Kainate was given IP at a dose of 20 mg/kg to the seven clavulanate treated animals and 13 saline vehicle treated animals. Over the next 60 minutes, animals were observed for seizure activity. Sixty minutes post kainate treatment the animals were given another IP injection (10 μg/kg) of clavulanate or saline vehicle. Animals that survived kainate treatment were sacrificed seven days later and their brains examined for histological changes in the hippocampus. Ten untreated control animals of the same weight and age as the experimental animals were sacrificed and their hippocampal morphology used as a standard for comparison.

Tissue Processing and Cresyl Violet Staining

On completion of the experiment, animals were anesthetized by IP injection of 10 mg/kg pentobarbital sodium, then perfused transcardially with 100 ml of PBS at pH 7.4, follow by 250 ml of 4% paraformaldehyde in PBS over a 5 min period. The brains were removed from the skull and post-fixed by immersion in the same fixative solution for 4 h, then transferred to 30% sucrose in PBS. After equilibration in the sucrose solution, coronal sections were cut using the cryocut at a thickness of 40 μm into storing solution and stored at 4° C. prior to staining. All stains are carried out on a 1 in 5 series of sections. One series of sections from each brain was stained using the general neuronal stain cresyl violet as follows. Sections were mounted onto gelatin-coated microscope slides and allowed to dry at room temperature overnight. Slides were then hydrated by 5 minute immersion in descending series of alcohols (90%, 80%, and 70% ethanol), then 30 minute immersion in distilled water. Staining was carried out by 3 min immersion in cresyl violet solution (5% in 0.1 M sodium acetate buffer, pH 3.5). Differentiation of the stain and dehydration was carried out in an ascending series of alcohols (70%, 80%, 90%, 95%, and 100% ethanol) before cleaning in xylene and cover slipping using Histomount mounting medium.

Quantitation of Data and Statistical Analysis

To evaluate the effects of clavulanate against kainate induced neuronal damage, the measurement of neuronal number was performed using an image analyzing system equipped with a computer-based CCD camera (Multiscan, Fullerton, Calif.). The number of cresyl violet positive neurons was counted in a 1 mm diameter of hippocampus in five sections per animal. The number of cresyl violet positive neurons was compared to that of the control group. Data are expressed as the mean±SEM. The data were evaluated by a one-way ANOVA SPSS program and the means assessed using Duncan's multiple-range test. Statistical significance was considered at P<0.05.

Results

Animals given clavulanate showed a longer onset to seizure and mild seizure activity as compared to controls given saline only. Respectively, six of the kainate+saline group died within 24 hrs of kainate treatment. But, clavulanate+kainate group showed no fatality. Table 15 tabulates the seizure rating scale (Sperk et al., 1983). Seizure rate was measured on 60-120 min after kainate treatment.

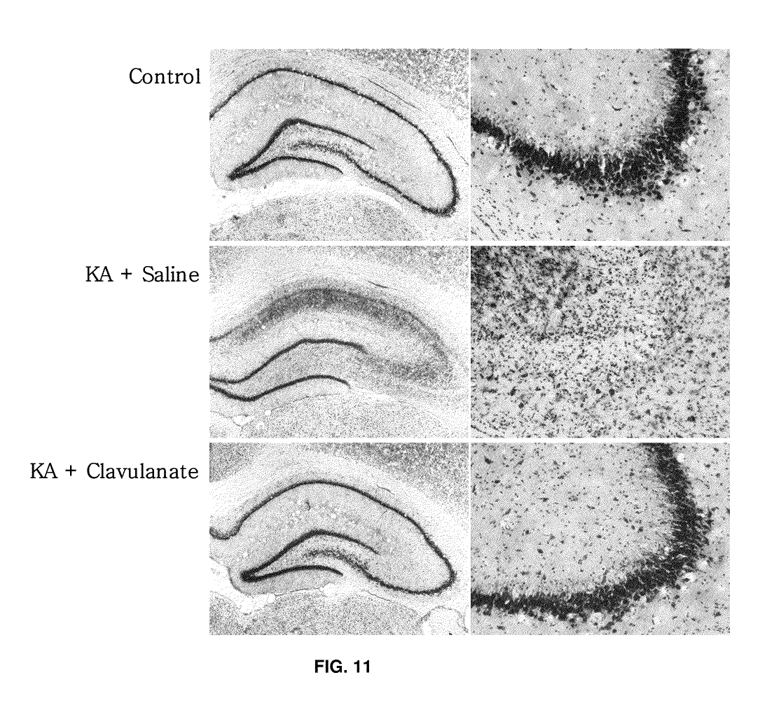

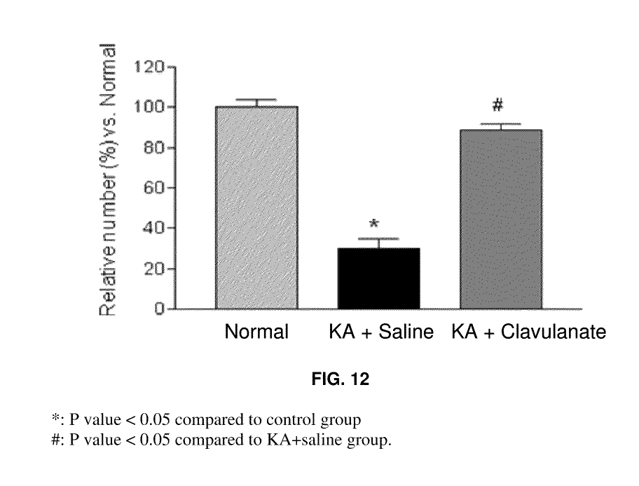

Animals given clavulanate showed significant neuroprotective effect on kainate induced hippocampal cell death. FIG. 11 shows the effect of clavulanate on kainate (KA) induced hippocampal neurotoxicity. The number of neurons in CA3 was significantly decreased in KA treated rats (KA+saline). clavulanate treatment on KA treated rats showed strong neuroprotective effect on CA3 region. In the kainate-saline treated group, cresyl violet positive CA3 cells in the stratum pyramidale were significantly decreased 7 days after kainate treatment. In this group, cresyl violet positive neurons in the stratum pyramidale were 29.7% compared to the normal group. In the clavulanate treated group, 88.7% of pyramidal neurons were positive to cresyl violet. FIG. 12 shows cresyl violet staining in the CA3 region in normal, kainate+saline, and kainate+clavulanate treated groups. KA+saline group showed significant decrease of cresyl violet positive neurons compared to normal group. In the clavulanate treated group, abundant cresyl violet positive neurons were observed in the stratum pyramidale in the CA3 region. Each column represents the Mean±S.E.M. (*: P value

<0.05 compared to control group. #: P value<0.05 compared to KA+saline group.) Also, clavulanate treated rats appeared to have normal morphology of neurons in CA3 as compared to animals treated with kainate-saline.

TABLE 15

| | | |
|---|---|---|
| Group | Survival rate (%) | Seizure rate |

| | | | |
|---|---|---|---|
| Kainate + saline | 53.8 | (7/13) | 4 |
| Kainate + clavulanate | 100 | (7/7) | 0-1 |
| Saline | 100 | (10/10) | 0 |

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

What is claimed is:

1. A method of treating a neurodegenerative disease comprising orally administering a stable oral formulation comprising a clavulanate in a therapeutically effective amount;
wherein the clavulanate is selected from the group consisting of clavulanic acid, a clavulanic acid derivative or a pharmaceutically acceptable salt of clavulanic acid.

2. A method of providing neuroprotection comprising orally administering a stable oral formulation comprising a clavulanate in a therapeutically effective amount;
wherein the clavulanate is selected from the group consisting of clavulanic acid, a clavulanic acid derivative or a pharmaceutically acceptable salt of clavulanic acid.

3. A method of preventing neuronal cell loss or death comprising orally administering a stable oral formulation comprising a clavulanate in a therapeutically effective amount;
wherein the clavulanate is selected from the group consisting of clavulanic acid, a clavulanic acid derivative or a pharmaceutically acceptable salt of clavulanic acid.

4. The method of claim 2, wherein neuroprotection comprises preventing cell loss or cell death from a neurodegenerative disease.

5. The method claim 1, wherein the neurodegenerative disease is selected from the group consisting of Parkinson's disease, Alzheimer's disease, and multiple sclerosis.

6. The method claim 1, wherein the clavulanate is potassium clavulanate.

7. The method of claim 1, wherein the oral formulation is in the form of a tablet, capsule, pill, troche, solution, suspension, buccal or sublingual tablet, orally disintegrating tablet, thin film or powder.

8. The method claim 1, wherein the formulation is an extended-release composition which releases the clavulanate for at least about 4 hours.

9. The method claim 1, wherein the formulation is an immediate-release composition which releases the clavulanate in less than about 0.5 hours.

10. The method of claim 6, wherein the potassium clavulanate is potassium clavulanate powder or potassium clavulanate as a 1:1 mixture with silicon dioxide or microcrystalline cellulose.

11. The method claims 1, wherein the formulation is prepared by the process of mixing the clavulanate with at least one excipient;
granulating the mixture of clavulanate and the at least one excipient; and
lyophilizing the granulated mixture of clavulanate and the at least one excipient.

12. The method claim 1, wherein the formulation is administered in an amount that provides from about 0.001 mg/kg/day to about 1.0 mg/kg/day of clavulanate.

13. The method of claim 1, wherein the formulation is administered in a single daily dose.

14. The method of claim 1, wherein the formulation is administered in multiple doses.

15. The method of claim 1, wherein treating comprises reducing the frequency, onset time or severity of seizures or tremors.

16. The method claim 1, wherein treating comprises reducing memory loss.

17. The method claim 1, wherein treating comprises reducing neuronal cell death.

18. The method of any one of claims 1, wherein the formulation comprises one or more of a matrix; a filler; a glidant; and a lubricant.

19. The method of claim 18, wherein the matrix is selected from the group consisting of Methocel K100LV Prem CR, Eudragit S100, Carbopol 971P, Carbopol 974P, methyacrylate copolymer type A and methacrylate copolymer type B and mixtures thereof; the filler is selected from the group consisting of anhydrous lactose, Avicel PH-112, Avicel PH-113, Isomalt, and mixtures thereof; the glidant is Carbosil and the lubricant is at least one of magnesium stearate and talc.

20. The method of claims 4, wherein the neurodegenerative disease is selected from the group consisting of Parkinson's disease, Alzheimer's disease, and multiple sclerosis.
